(19) United States
(12) Patent Application Publication
(10) Pub. No.: US 2024/0034883 A1
(43) Pub. Date: Feb. 1, 2024

(54) RIGID LINKERS TO PROMOTE J-PACKING AND ENHANCE Kr

(71) Applicant: Boise State University, Boise, ID (US)

(72) Inventors: Olga A. Mass, Boise, ID (US); William B. Knowlton, Boise, ID (US)

(21) Appl. No.: 18/361,055

(22) Filed: Jul. 28, 2023

Related U.S. Application Data
(60) Provisional application No. 63/369,807, filed on Jul. 29, 2022.

(51) Int. Cl. C09B 47/00; C12Q 1/6816

(52) U.S. Cl. CPC C09B 47/00; C12Q 1/6816

(57) ABSTRACT

Dye aggregates that are templated to deoxyribonucleic acid (DNA) can exhibit different packing arrangements and, as an optically active medium, spectral properties of interest. Steric effects and properties of the linker that tethers the dye to a DNA scaffold (a simple nucleotide oligomer or more complex architectures) can used to fine tune the packing arrangement of the dyes to obtain desirable spectral properties. To gain access to J-aggregates (head-to-tail packing arrangement) as a source of fluorescent materials, the present disclosure is directed to two strategies that utilize rigid linkers to promote J-packing. Devices and methods of use also are disclosed herein.

### CROSS REFERENCE TO RELATED APPLICATIONS

This application claims priority under 35 U.S.C. § 119 to provisional application Ser. No. 63/369,807, filed Jul. 29, 2022, herein incorporated by reference in its entirety.

### STATEMENT REGARDING FEDERALLY SPONSORED RESEARCH OR DEVELOPMENT

This invention was made with government support under Award Number DE-SC0020089, awarded by the U.S. Department of Energy. The government has certain rights in the invention.

### TECHNICAL FIELD

The invention relates generally to dyes comprising a chromophore or fluorophore bound to a nucleotide architecture. More specifically, the invention relates to the use of rigid linkers to promote J-packing of the dye chromophores and/or fluorophores and enhancing the radiative rate, kᵣ.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

An exciton is the name for the energy packet that resides on a dye, sometimes called a chromophore or fluorophore, when it is in its excited state. When two dyes are very close to each other such that the transition dipole-dipole coupling is weak to strong, the energy of an excited chromophore can be shared with a neighboring chromophore without energy loss (coherent energy transfer), in contrast to the usual Förster resonance energy transfer (FRET) where the dipole-dipole coupling is very weak and energy loss occurs in the transfer (incoherent energy transfer). The packet of energy can be exchanged in a wave like manner back and forth between the two molecules. The energy packet, in this sense, acts like a quantum mechanical particle that can become delocalized or spread out over an aggregate of chromophores just like an electron can spread out its wave function over an entire molecule (molecular orbital). The Davydov splitting (i.e., splitting of the monomeric electronic energy levels) and the circular dichroism (CD) spectra seen in chromophore aggregates is a manifestation of this delocalization. Davydov splitting is analogous to the splitting of orbitals into bonding and antibonding orbitals when two atoms are brought close together to form a molecule. Similar to passing the exciton (energy) from chromophore to chromophore, aggregates may also be used to pass an exciton (energy) from aggregate to aggregate.

When two or more dyes are brought very close to each other or aggregate in high concentrations in solution, excitonic quantum coherent behavior (e.g., large Davydov splitting, exchange narrowing, circular dichroism, Cotton effects, or Stokes shifting) can be observed. The excitonic quantum coherent behavior of the changes depends on the type of stacking of the dyes. In general dyes will stack one of three ways: J-like, H-like, and oblique.

In J-like stacking, the transition dipole moments of two or more chromophores are aligned more or less end-to-end and have a narrowing of the Stokes shift, usually exhibit a red shift in absorption due to the selection rules for dye aggregates allowing access to the lowest excited state and exhibit radiative emission due to Kasha's rule. In H-like stacking, where the transition dipole moments of two or more dyes are aligned in a more or less parallel arrangement, the Stokes shift is widened, there is usually a blue shift in absorption as the selection rules allow access to a higher excited energy state when compared to the monomer, and the dyes have a very low amount of radiative emission, if at all, as energy loss occurs primarily via non-radiative pathways (i.e., Kasha's rule, see M. Kasha, Characterization of electronic transitions in complex molecules, Discussions of the Faraday Society, 9 (0), 14-19 (1950), M. Kasha, 1963, Energy transfer mechanisms and the molecular exciton model for molecular aggregates, Radiation Research, 20(1): 55-70; E.G. McRae and M. Kasha, The molecular exciton model, in Physical processes in radiation biology, edited by L. Augestein et al. (Academic Press, 1964), pp. 23-44; and M. Kasha et al., 1965, The exciton model in molecular spectroscopy, Pure and Applied Chemistry, 11(3-4): 371-392, herein incorporated in their entirety). Oblique stacking, sometimes called J/H-like, is a mix of J-like and H-like characteristics, in which Davydov splitting is observed in absorption whereby excitation to both the lowest and highest excited state energy levels, as compared to the monomer, are both allowed due to the selection rules. Radiative emission occurs only from the lowest energy state following Kasha's rule. Coupled dyes that return to their ground state radiatively offer a number of desirable optical properties.

However, due to the structural nature of dyes, most DNA-templated dyes tend to configure into H-like stacking. Therefore, creating J-like stacking is challenging as materials and strategies are lacking for the promotion or fixing of the dyes into J-like stacking.

Excited dyes will also lose energy through multiple other non-radiative paths. For example, a dye may lose energy through emitting heat or infrared light. A dye may also lose energy to surrounding molecules through vibrational coupling. These non-radiative paths may negatively impact the lifetime of the excitation of the dye, resulting in a loss of fluorescence from the dye.

Therefore, there continues to be a need to develop ways to promote dyes to take the end-to-end J-like stacking configuration to gain access to the desired optical properties.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

Applicants have created compositions of one or more dyes attached to a nucleotide architecture that promotes J-like stacking of the dyes.

In an aspect, linkers between a dye and nucleotide are used to limit the conformational rotation of the dye. The linkers are preferably rigid to maintain desired orientation of dyes' transition dipole moments (end-to-end orientation of transition dipole moments) when attached to DNA. In an embodiment, the linker is a C₂ to C₃₀ alkyne, more preferably a C₂ to C₂₀ alkyne, and even more preferably a C₂ to C₈ alkyne. In various embodiments, the linker may be linear or cyclical and may be further substituted. In some embodiments, it is preferred to link the dye through the linker to a thymine or thymine analog. In some preferred embodiments, the dye is tetrapyrrole. In some preferred embodiments, the tetrapyrrole is chlorin or bacteriochlorin.

In further embodiments, the steric motion of the dyes is limited by substituting the dye with a compound that inhibits torsional rotation of the dyes. This may promote J-like stacking.

In preferred embodiments, the compound is an aryl. In more preferred embodiments, the compound is mesityl. The dye may have multiple aryl substitutions.

In another aspect, two or more dyes are covalently bridged in a non-conjugating fashion. In a preferred embodiment, the dyes are bridged to configure their transition dipole moments end-to-end. In preferred embodiments, the bridge is bicyclic or a carbon cage. In preferred embodiments, the bridge is a bicyclopentane, bicyclooctane, triptycene, cubane, or a diamondoid. In a more preferable embodiment, the bridge is an adamantane. In further examples, the bridge may be substituted.

The dyes linked to a nucleotide may be used as part of any compositions, system, or method where J-like stacking is desired. In some embodiments, the dye linked to a nucleotide may be a photosensitizer, an imager, or used for detection. The linked nucleotide may be part of a nucleotide architecture, wherein the nucleotide architecture is a delivery system. In other embodiments, the dyes linked to a nucleotide may be used as part of optical systems or devices.

While multiple embodiments are disclosed, still other embodiments of the present invention will become apparent to those skilled in the art from the following detailed descriptions, which show and describe illustrative embodiments of the invention. Accordingly, the figures and detailed description are to be regarded as illustrative in nature and not restrictive.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1A shows helical arrangement of chlorins (Chls) attached to dsDNA via acetylene linker in adjacent fashion (i.e., attached to the nucleotides of the same strand) (bottom; top view).

FIG. 1B shows structures of bis-Chl and mono-Chl.

FIG. 2 shows three level site basis states.

FIG. 3 shows Frenkel excitation Hamiltonian energies and excitation energy schematics.

FIG. 4 shows a graphical representation of the quantities ε_{m,α}^{v} and D_{m,α}for vibronic mode α of dye m.

As shown in FIG. 5A, in bis-BC-1, two bacteriochlorins dyes are linked via cyclo[1.1.1]pentane bridge.

As shown in FIG. 5B, in bis-BC-2, two bacteriochlorins dyes are linked via spiro[4.4]nonane bridge.

FIG. 5C shows a general chemical structure of a bis-dye where two hydroporphyrin dyes (i.e. chlorin, bacteriochlorin, or their combination) are covalently bridged in a non-conjugating fashion through a non-conjugating bridge. The transition dipole moments (double-headed arrows) are in an end-to-end configuration. Hydroporphyrins can carry a variety of substituents.

Examples of non-conjugating bridges are shown in FIG. 5D. All bridges can be used interchangeably with any of bis-BC-1, bis-BC-2, and bis-BC-3.

FIG. 6 shows an optimized geometry of Bis-BC-1 via DFF-calculations. Long wavelength (QyQy) transition dipole moments of each constituent dye (monomer) are shown as double-headed arrows. Point charges r_1 and r_2 are labeled as cyan dots on the far left and right of the super dye, and point charges s_1 and s_2 are labeled as orange dots on the inner left and right of the super dye.

FIG. 7 shows an optimized geometry of Bis-BC-2 via DFT-calculations. Long wavelength (Q_{y}) transition dipole moments of each constituent dye (monomer) are shown as double-headed arrows. Point charges r₁ and r₂ are labeled as cyan dots on the far left and right of the super dye, and point charges s₁ and s₂ are labeled as orange dots on the inner left and right of the super dye.

FIG. 8 shows a synthetic scheme for synthesis of bis-BC-1.

FIG. 9 shows a synthetic route to bis-BC-3.

FIG. 10A shows DFT-calculated molecular orbitals HOMO-1, HOMO, LUMO, and LUMO+1 of bis-BC-3 in vacuum.

FIG. 10B shows DFT-calculated molecular orbitals HOMO-1, HOMO, LUMO, and LUMO+1 of bis-BC-3 in toluene show no electron density on the methylene carbons of the cyclobutene bridge, hence, confirming the non-conjugating nature of this bridge.

An artisan of ordinary skill in the art need not view, within isolated figure(s), the near infinite distinct combinations of features described in the following detailed description to facilitate an understanding of the present disclosure.

### DETAILED DESCRIPTION

In the following detailed description, reference may be made to the accompanying drawings, schemes, and structures which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The embodiments of this disclosure are not limited to any specific compositions and methods which can vary and are understood by skilled artisans. It is further to be understood that all terminology used herein is for describing particular embodiments only and is not intended to be limiting in any manner or scope. For example, as used in this specification and the appended claims, the singular forms “a,” “an”, and “the” can include plural referents unless the content clearly indicates otherwise. Further, all units, prefixes, and symbols may be denoted in its SI accepted form.

Numeric ranges recited within the specification are inclusive of the numbers within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range (i.e., 1 to 5 includes 1, 1.5, 2, 2.75, 3, 3.80, 4, and 5).

So that the present disclosure may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

Other than in the operating examples, or where otherwise indicated, all numbers expressing quantities of ingredients or reaction conditions used herein are to be understood as being modified in all instances by the term “about”.

As used herein, the term “about” modifying the quantity or quality of an ingredient in the compositions of the invention or employed in the methods of the invention refers to variation in the numerical quantity that can occur, for example, through typical measuring errors; through inadvertent error in these procedures; through differences in the manufacture, source, or purity of the ingredients employed to make the compositions or carry out the methods; and the like. Whether or not modified by the term “about”, the claims include equivalents to the quantities.

“Non-covalent” refers to any molecular interactions that are not covalent—i.e., the interaction does not involve the sharing of electrons. The term includes, for example, electrostatic, π-effects, van der Waals forces, and hydrophobic effects. “Covalent” refers to interactions involving the sharing of one or more electrons.

As used herein, a “linker” is a compound that bridges or connects two or more molecules. The two or more molecules may share other bonds in addition to the linker, for example ionic bonds, polar covalent bonds, or covalent bonds.

As used herein, a “brick” is a structural unit. A brick may be of any shape or size. The main body of a brick may be of any material composition. An example of a brick is a “nucleotide brick,” which is a structural unit where the body of the brick is made of a nucleotide oligomer. An example of a nucleotide brick is a “DNA brick,” which is a nucleotide brick where the body of the brick is made of a DNA oligomer.

As used herein, a “nucleotide” is any nucleoside bonded to a phosphate group. The nucleoside may be natural, including but not limited to, any of cytidine, uridine, adenosine, guanosine, thymidine, inosine (hypoxanthine), or uric acid; or synthetic, including but not limited to methyl-substituted phenol analogs, hydrophobic base analogs, purine/pyrimidine mimics, icoC, isoG, thymidine analogs, fluorescent base analogs, or X or Y synthetic bases. Alternatively, a nucleotide may be abasic, such as but not limited to 3-hydroxy-2-hydroxymethyl-tetrahydrofuran, which act as a linker group between two phosphate groups in an oligonucleotide and lacks a base or may be a nucleotide analog.

As used herein, “nucleotide duplex” is when two strands of complement nucleotide oligomers complementary bind to each other. The two strands may be part of the same nucleotide molecule or separate nucleotide molecules.

As used herein, “nucleotide origami” is two or more nucleotide bricks, where one brick is a “scaffold” and provides the main body of the overall structure and is bound by one or more “staple(s).”

As used herein, a “scaffold” is a single stranded nucleotide brick rationally-designed to self-assemble into hairpin loops, helical domains, and locking domains. The scaffold may use staples to direct the folding and to hold the final shape. Alternatively, the scaffold may use intrinsic self-complementary pairing to hold the final shape.

As used herein, a “staple” or “staple strand” is a nucleotide brick which pairs with a longer main body brick in nucleotide origami to help fold the main body brick into the desired shape.

As used herein, a “nanobreadboard,” “breadboard,” or “template” is a total or final structure of a DNA structure or shape. For example, a mobile or immobile 4-arm junction, DNA origami happy face, rectangular brick, or double stranded DNA (dsDNA) in its final structure.

As used herein, an “architecture” is a one-, two-, or three-dimensional structure built using one or more bricks. As used herein, a “nucleotide architecture” is a one-, two-, or three-dimensional structure built using one or more nucleotide bricks. Examples include nucleotide origami or molecular canvases.

As used herein, “self-assembly” refers to the ability of nucleotides to adhere to each other, in a sequence-specific manner, in a predicted manner and without external control.

As used herein, “sufficiently close” and “nanospaced” refers to a distance between two chromophores that allows one chromophore, when excited, to create an exciton whose excitation energy is shared with a second chromophore without a loss of energy (i.e., coherent energy transfer).

As used herein, a “breadboard” refers to reusable solderless device used to build an excitonic circuit. The breadboard allows for temporary placement of different solutions, such as solutions containing chromophore bound nucleotide architectures, in different arrangements.

As used herein, Förster resonance energy transfer (FRET), fluorescence resonance energy transfer (FRET), resonance energy transfer (RET), or electronic energy transfer (EET) refers to energy transfer between two light-sensitive molecules (donor and acceptor chromophores) or aggregates thereof in an incoherent process in which energy loss occurs.

As used herein, the terms “dye aggregate,” “chromophore aggregate,” or “aggregate” are used interchangeably unless otherwise specified. Aggregates are also referred to as mers of dyes. A dye aggregate comprised of two dyes is called a dimer, composed of three dyes is called a trimer, composed of four dyes is called a tetramer, and so on.

As used herein, the term “dye” refers to a molecule comprising a “chromophore” or a “fluorophore.” As the chromophore or fluorophore may comprise the entire molecule, “dye”, “chromophore”, and “fluorophore” may be used interchangeably with each other unless otherwise specified.

As used herein, “substituted” refers to an organic group as defined below (i.e., an alkyl group) in which one or more bonds to a hydrogen atom contained therein are replaced by a bond to non-hydrogen or non-carbon atoms. Substituted groups also include groups in which one or more bonds to carbon(s) or hydrogen(s) atom may be replaced by one or more bonds, including double or triple bonds, to a heteroatom. Thus, a substituted group is substituted with one or more substituents, unless otherwise specified. A substituted group can be substituted with 1, 2, 3, 4, 5, 6, or more substituents.

Substituted ring groups include rings and ring systems in which a bond to a hydrogen atom is replaced with a bond to a carbon atom. Therefore, substituted cycloalkyl, aryl, heterocyclyl, and heteroaryl groups may also be substituted with substituted or unsubstituted alkyl, alkenyl, and alkynyl groups as defined herein.

As used herein, the term “alkyl” or “alkyl groups” refers to saturated hydrocarbons having one or more carbon atoms, including straight-chain alkyl groups (i.e., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (i.e., cyclopropyl, cyclopentyl, cyclohexyl, cycloheptyl, cyclooctyl, etc.), branched-chain alkyl groups (i.e., isopropyl, tert-butyl, sec-butyl, isobutyl, etc.), and alkyl-substituted alkyl groups (i.e., alkyl-substituted cycloalkyl groups and cycloalkyl-substituted alkyl groups).

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan.

Alkenyl groups or alkenes are straight chain, branched, or cyclic alkyl groups having two to about 30 carbon atoms, and further including at least one double bond. In some embodiments, an alkenyl group has from 2 to about 30 carbon atoms, or typically, from 2 to 10 carbon atoms. Alkenyl groups may be substituted or unsubstituted. For a double bond in an alkenyl group, the configuration for the double bond can be a trans or cis configuration. Alkenyl groups may be substituted similarly to alkyl groups.

Alkynyl groups are straight chain, branched, or cyclic alkyl groups having two to about 30 carbon atoms, and further including at least one triple bond. In some embodiments, an alkynyl group has from 2 to about 30 carbon atoms, or typically, from 2 to 10 carbon atoms. Alkynyl groups may be substituted or unsubstituted. Alkynyl groups may be substituted similarly to alkyl or alkenyl groups.

As used herein, the terms “alkylene”, “cycloalkylene”, “alkynylides”, and “alkenylene”, alone or as part of another substituent, refer to a divalent radical derived from an alkyl, cycloalkyl, or alkenyl group, respectively, as exemplified by —CH2CH2CH2—. For alkylene, cycloalkylene, alkynylene, and alkenylene groups, no orientation is implied.

The term “ester” as used herein refers to —R30COOR31 group. R30 is absent, a substituted or unsubstituted alkylene, cycloalkylene, alkenylene, alkynylene, arylene, aralkylene, heterocyclylalkylene, or heterocyclylene group as defined herein. R31 is a substituted or unsubstituted alkyl, cycloalkyl, alkenyl, alkynyl, aryl, aralkyl, heterocyclylalkyl, or heterocyclyl group as defined herein.

The term “amine” (or “amino”) as used herein refers to —R32NR33R34 groups. R32 is absent, a substituted or unsubstituted alkylene, cycloalkylene, alkenylene, alkynylene, arylene, aralkylene, heterocyclylalkylene, or heterocyclylene group as defined herein. R33 and R34 are independently hydrogen, or a substituted or unsubstituted alkyl, cycloalkyl, alkenyl, alkynyl, aryl, aralkyl, heterocyclylalkyl, or heterocyclyl group as defined herein.

The term “amine” as used herein also refers to an independent compound. When an amine is a compound, it can be represented by a formula of R32′NR33′R34′ groups, wherein R32′, R33′, and R34 are independently hydrogen, or a substituted or unsubstituted alkyl, cycloalkyl, alkenyl, alkynyl, aryl, aralkyl, heterocyclylalkyl, or heterocyclyl group as defined herein.

The term “alcohol” as used herein refers to —R35OH groups. R35 is absent, a substituted or unsubstituted alkylene, cycloalkylene, alkenylene, alkynylene, arylene, aralkylene, heterocyclylalkylene, or heterocyclylene group as defined herein.

The term “carboxylic acid” as used herein refers to —R36COOH groups. R36 is absent, a substituted or unsubstituted alkylene, cycloalkylene, alkenylene, alkynylene, arylene, aralkylene, heterocyclylalkylene, or heterocyclylene group as defined herein.

The term “ether” as used herein refers to —R37OR38 groups. R37 is absent, a substituted or unsubstituted alkylene, cycloalkylene, alkenylene, alkynylene, arylene, aralkylene, heterocyclylalkylene, or heterocyclylene group as defined herein. R38 is a substituted or unsubstituted alkyl, cycloalkyl, alkenyl, alkynyl, aryl, aralkyl, heterocyclylalkyl, or heterocyclyl group as defined herein.

### Oligonucleotides

The dyes disclosed herein may be tethered to one, two, or more oligonucleotides. As used herein, an oligonucleotide can contain all the natural nucleotides found in nature or one, more, or all modified or synthetic nucleotides, in addition to the natural nucleotides and the nucleotides containing the donor, acceptor, or photochromic moiety. A modified or synthetic nucleotide in the oligonucleotides can differ from a natural occurring nucleotide in its base, sugar, and/or backbone moiety.

The oligonucleotides disclosed herein can be, but are not limited to, a peptide nucleic acid (PNA), a locked nucleic acid (LNA), an unlocked nucleic acid (UNA), a bridged nucleic acid polymer, or combination thereof.

PNA oligomers can show greater specificity in binding to complementary DNAs, with a PNA/DNA base mismatch being more destabilizing than a similar mismatch in a DNA/DNA duplex.

A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. LNA polymer are synthesized chemically and are commercially available. The locked ribose conformation enhances base stacking and backbone pre-organization. This significantly increases the hybridization properties (melting temperature) of oligonucleotides.

Bridged nucleic acids (BNAs) are modified RNA nucleotides. They are sometimes also referred to as constrained or inaccessible RNA molecules. BNA monomers can contain a five-membered, six-membered, or even a seven-membered bridged structure with a “fixed” C3′-endo sugar puckering. The bridge is synthetically incorporated at the 2′, 4′-position of the ribose to afford a 2′, 4′-BNA monomer. The monomers can be incorporated into oligonucleotide polymeric structures using standard phosphoramidite chemistry. BNAs are structurally rigid oligo-nucleotides with increased binding affinities and stability.

The nucleobase making up the oligomer may be natural, including but not limited to, any of cytosine, uracil, adenine, guanine, thymine, hypoxanthine, or uric acid; or synthetic, including but not limited to methyl-substituted phenol analogs, hydrophobic base analogs, purine/pyrimidine mimics, icoC, isoG, thymidine analogs, fluorescent base analogs, or X or Y synthetic bases. Alternatively, a nucleotide may be abasic, such as but not limited to 3-hydroxy-2-hydroxymethyl-tetrahydrofuran, or alternatively a nucleotide analog may be used.

Non-limiting examples of synthetic nucleobases and analogs include, but are not limited to methyl-substituted phenyl analogs, such as but not limited to mono-, di-, tri-, or tatramethylated benzene analogs; hydrophobic base analogs, such as but not limited to 7-propynyl isocarbostyril nucleoside, isocarbostyril nucleoside, 3-methylnapthalene, azaindole, bromo phenyl derivates at positions 2, 3, and 4, cyano derivatives at positions 2, 3, and 4, and fluoro derivates at position 2 and 3; purine/pyrimidine mimics, such as but not limited to azole hetercyclic carboxamides, such as but not limited to (1H)-1,2,3-triazole-4-carboxamide, 1,2,4-triazole-3-carboxamide, 1,2,3-triazole-4-carboxamide, or 1,2-pyrazole-3-carboxamide, or heteroatom-containing purine mimics, such as furo or theino pyridiones, such as but not limited to furo[2,3-c]pyridin-7(6H)-one, thieno[2,3-c]pyridin-7(6H)-one, furo[2,3-c]pyridin-7-thiol, furo[3,2-c]pyridin-4(5H)-one, thieno[3,2-c]pyridin-4(5H)-one, or furo[3,2-c]pyridin-4-thiol, or other mimics, such as but not limited to 5-phenyl-indolyl, 5-nitro-indolyl, 5-fluoro, 5-amino, 4-methylbenzimidazole, 6H,8H-3,4-dihydropropyrimido[4,5-c][1,2]oxazin-7-one, or N⁶-methoxy-2,6-diaminopurine; isocytosine, isoquanosine; thymidine analogs, such as but not limited to 5-methylisocyto sine, difluorotoluene, 3-toluene-1-β-D-deoxyriboside, 2,4-difluoro-5-toluene-1-β-D-deoxyriboside, 2,4-dichloro-5-toluene-1-β-D-deoxyriboside, 2,4-dibromo-5-toluene-1-β-D-deoxyribo side, 2,4-diiodo-5-toluene-1-β-D-deoxyriboside, 2-thiothymidine, 4-Se-thymidine; or fluorescent base analogs, such as but not limited to 2-aminopurine, 1,3-diaza-2-oxophenothiazine, 1,3-diaza-2-oxophenoxazine, pyrrolo-dC and derivatives, 3-MI, 6-MI, 6-MAP, or furan-modified bases.

Non-limiting examples of nucleotide analogs include, but are not limited to, phosporothioate nucleotides, 2′-0-methyl ribonucleotides, 2′-0-methoxy-ethyl ribonucleotides, peptide nucleotides (PNA), N3′-P5′ phosphoramidate, 2′-fluoro-arabino nucleotides, locked nucleotides (LNA), unlocked nucleotides (UNA), bridge nucleotides (BNA), morpholino phosphoramidate, cyclohexene nucleotides, tricyclo-deoxynucleotides, or triazole-linked nucleotides.

A modified nucleotide can be, but is not limited to, d5SICS and dNaM that base pair with each other and dTPT3 also base pairs with dNaM (Floyd Romesberg), 2-amino-8-(2-thienyl)purine that base-pairs with pyridine-2-one (y), 7-(2-thienyl)imidazo[4,5-b]pyridine (Ds) that base-pairs with pyrrole-2-carbaldehyde (Pa), and Ds that base pairs with 4-[3-(6-aminohexanamido)-1-propynyl]-2-nitropyrrole (Px).

The oligonucleotide can be a single strand, in which one or more dyes can fold into such a conformation, so that the dyes are close enough to each other, so that a photochromic Förster resonance energy transfer (pcFRET) can happen between the dyes.

In Förster resonance energy transfer (FRET), fluorescence resonance energy transfer (FRET), resonance energy transfer (RET), or electronic energy transfer (EET) energy transfer between two light-sensitive molecules, such as between two or more dyes occurs when a first dye, initially in its electronic excited state, transfers energy to a second dye through nonradiative dipole-dipole coupling. The dipole-dipole coupling occurring in FRET is classified as a very weak coupling. The efficiency of this energy transfer is inversely proportional to the sixth power of the distance between the first and second chromophore, resulting in a loss of energy during the transfer. In FRET, the excited chromophore, or donor chromophore, emits a virtual or undetectable photon that is instantly absorbed by a second chromophore, the acceptor chromophore. FRET does not change the absorbance spectrum or emittance spectrum of the individual chromophores involved in the energy transfer.

While FRET is the transfer of energy between two chromophores, a system of chromophores, comprising more than two chromophores, may each use FRET to transfer energy from one chromophore to the next along the system as long as the absorbance spectrum of the acceptor chromophore is within the emittance spectrum of the donor chromophore. Therefore, each chromophore may act as a donor chromophore and acceptor chromophore in the system.

Exciton transfer or sharing, or the molecular excitation model, differs from FRET in several ways. While FRET is a very weak dipole-dipole coupling that transfers energy through a virtual photon, exciton transfer or sharing requires a weak to strong coupling of the dipoles of two or more chromophores and shares excitons within the aggregate. When two or more chromophore dipoles are weakly to strongly coupled, excitons may delocalize over the two or more chromophores. Therefore, the energy captured by the exciton is shared within the aggregate and so energy is not lost when the energy is transferred from chromophore to chromophore through the aggregate.

Also, unlike FRET, a weakly to strongly coupled aggregate of two or more chromophores may seemingly act as a single chromophore. This may be measured by detecting a change in the absorbance or emission spectrum of the system.

The structure may be a duplex including two or more mostly matching or complementary oligonucleotides. In this situation, one of the two or more oligonucleotides can contain one of or all the dyes, and the other can contain the rest.

In some embodiments, two or more oligonucleotides form one duplex. In some embodiments, two or more oligonucleotides form two or more duplexes.

### Nucleotide Architecture

The chromophores may optionally be attached to more complicated nucleotide architectures. Nucleotide nanotechnology can be used to form complicated one-, two-, and three-dimensional architectures. The nucleotide architectures may comprise of one or more nucleotide bricks. The nucleotide bricks are designed to use the Watson-Crick pairing of the nucleotides to cause the bricks to self-assemble into the final and predictable architectures. Any method of designing the architectures and self-assembly may be used, such as but not limited to nucleotide origami, nucleotide brick molecular canvases, single stranded tile techniques, or any other method of nucleotide folding or nanoassembly such as, but not limited to, using nucleotide tiles, nucleotide scaffolds, nucleotide lattices, four-armed junction, double-crossover structures, nanotubes, static nucleotide structures, dynamically changeable nucleotide structures, or any other synthetic biology technique (as described in U.S. Pat. No. 9,073,962, U.S. Pub. Nos. 2017/0190573, 2015/0218204, 2018/0044372, or International Publication Number WO 2014/018675, each of which is incorporated in its entirety by reference).

The nucleobase making up the bricks may be natural, including but not limited to, any of cytosine, uracil, adenine, guanine, thymine, hypoxanthine, or uric acid; or synthetic, including but not limited to methyl-substituted phenol analogs, hydrophobic base analogs, purine/pyrimidine mimics, icoC, isoG, thymidine analogs, fluorescent base analogs, or X or Y synthetic bases. Alternatively, a nucleotide may be abasic, such as but not limited to 3-hydroxy-2-hydroxymethyl-tetrahydrofuran, or alternatively a nucleotide analog may be used.

Non-limiting examples of synthetic nucleobases and analogs include, but are not limited to methyl-substituted phenyl analogs, such as but not limited to mono-, di-, tri-, or tatramethylated benzene analogs; hydrophobic base analogs, such as but not limited to 7-propynyl isocarbostyril nucleoside, isocarbostyril nucleoside, 3-methylnapthalene, azaindole, bromo phenyl derivates at positions 2, 3, and 4, cyano derivatives at positions 2, 3, and 4, and fluoro derivates at position 2 and 3; purine/pyrimidine mimics, such as but not limited to azole hetercyclic carboxamides, such as but not limited to (1H)-1,2,3-triazole-4-carboxamide, 1,2,4-triazole-3-carboxamide, 1,2,3-triazole-4-carboxamide, or 1,2-pyrazole-3-carboxamide, or heteroatom-containing purine mimics, such as furo or theino pyridiones, such as but not limited to furo[2,3-c]pyridin-7(6H)-one, thieno[2,3-c]pyridin-7(6H)-one, furo[2,3-c]pyridin-7-thiol, furo[3,2-c]pyridin-4(5H)-one, thieno[3,2-c]pyridin-4(5H)-one, or furo[3,2-c]pyridin-4-thiol, or other mimics, such as but not limited to 5-phenyl-indolyl, 5-nitro-indolyl, 5-fluoro, 5-amino, 4-methylbenzimidazole, 6H,8H-3,4-dihydropropyrimido [4,5-c][1,2]oxazin-7-one, or N⁶-methoxy-2,6-diaminopurine; isocytosine, isoquanosine; thymidine analogs, such as but not limited to 5-methylisocyto sine, difluorotoluene, 3-toluene-1-β-D-deoxyriboside, 2,4-difluoro-5-toluene-1-β-D-deoxyriboside, 2,4-dichloro-5-toluene-1-β-D-deoxyriboside, 2,4-dibromo-5-toluene- 1-β-D-deoxyribo side, 2,4-diiodo-5-toluene-1-β-D-deoxyriboside, 2-thiothymidine, 4-Se-thymidine; or fluorescent base analogs, such as but not limited to 2-aminopurine, 1,3-diaza-2-oxophenothiazine, 1,3-diaza-2-oxophenoxazine, pyrrolo-dC and derivatives, 3-MI, 6-MI, 6-MAP, or furan-modified bases.

Non-limiting examples of nucleotide analogs include, but are not limited to, phosporothioate nucleotides, 2′-O-methyl ribonucleotides, 2′-O-methoxy-ethyl ribonucleotides, peptide nucleotides (PNA), N3′-P5′ phosphoramidate, 2′-fluoro-arabino nucleotides, locked nucleotides (LNA), unlocked nucleotides (UNA), bridge nucleotides (BNA), morpholino phosphoramidate, cyclohexene nucleotides, tricyclo-deoxynucleotides, or triazole-linked nucleotides.

A modified nucleotide can be, but is not limited to, d5SICS and dNaM that base pair with each other and dTPT3 also base pairs with dNaM (Floyd Romesberg), 2-amino-8-(2-thienyl)purine that base-pairs with pyridine-2-one (y), 7-(2-thienyl)imidazo[4,5-b]pyridine (Ds) that base-pairs with pyrrole-2-carbaldehyde (Pa), and Ds that base pairs with 4-[3(6-aminohexanamido)-1-propynyl]-2-nitropyrrole (Px).

The nucleotides can then be polymerized into oligomers. The design of the oligomers will depend on the design of the final architecture. Simple architectures may be designed by any methods. However, more complex architectures may be designed using software such as, but not limited to, caDNAno (as described at http://cadnano.org/docs.html, and herein incorporated by reference), to minimize errors and time. The user may input the desired shape of the architecture into the software and once finalized, the software will provide the oligomer sequences of the bricks to create the desired architecture.

In some embodiments the architecture is comprised of nucleotide brick molecular canvases, wherein the canvases are made of 1 to 5,000 nucleotide bricks comprising of nucleotide oligomers of 24 to 48 nucleotides and will self-assemble in a single reaction, a “single-pot” synthesis, as described in U.S. Pub. No. 2015/0218204. In more preferable embodiments, the canvases are made of 1 to 1,000 nucleotide bricks, from 1 to 750 nucleotide bricks, from 1 to 500 nucleotide bricks, or from 1 to 250 nucleotide bricks. In other embodiments, the oligomers comprise of 24 to 42 nucleotides, from 24 to 36 nucleotides, or from 26 to 36 nucleotides.

In another embodiment the architecture is made step wise using a serial fluidic flow to build the final shape as described in U.S. Pat. No. 9,073,962.

In some embodiments, the architecture is assembled using the origami approach. With a DNA origami approach, for example, a long scaffold nucleic acid strand is folded to a predesigned shape through interactions with relatively shorter staple strands. Thus, in some embodiments, a single-stranded nucleic acid for assembly of a nucleic acid nanostructure has a length of at least 500 base pairs, at least 1 kilobase, at least 2 kilobases, at least 3 kilobases, at least 4 kilobases, at least 5 kilobases, at least 6 kilobases, at least 7 kilobases, at least 8 kilobases, at least 9 kilobases, or at least 10 kilobases. In some embodiments, a single-stranded nucleic acid for assembly of a nucleic acid nanostructure has a length of 500 base pairs to 10 kilobases, or more. In some embodiments, a single-stranded nucleic acid for assembly of a nucleic acid nanostructure has a length of 7 to 8 kilobases. In some embodiments, a single-stranded nucleic acid for assembly of a nucleic acid nanostructure comprises the M13 viral genome. In some embodiments, the number of staple strands is less than about 500 staple strands, less than about 400 staple strands, less than about 300 staple strands, less than about 200 staple strands, or less than about 100 staple strands.

In some embodiments, the architecture is assembled from single-stranded tiles (SSTs) (see, e.g., Wei B. et al. Nature 485: 626, 2012, incorporated by reference herein) or nucleic acid “bricks” (see, e.g., Ke Y. et al. Science 388:1177, 2012; International Publication Number WO 2014/018675 A1 each of which is incorporated by reference herein). For example, single-stranded 2- or 4-domain oligonucleotides self-assemble, through sequence-specific annealing, into two- and/or three-dimensional nanostructures in a predetermined (e.g., predicted) manner. As a result, the position of each oligonucleotide in the nanostructure is known. In this way, a nucleic acid nanostructure may be modified, for example, by adding, removing or replacing oligonucleotides at particular positions. The nanostructure may also be modified, for example, by attachment of moieties, at particular positions. This may be accomplished by using a modified oligonucleotide as a starting material or by modifying a particular oligonucleotide after the nanostructure is formed. Therefore, knowing the position of each of the starting oligonucleotides in the resultant nanostructure provides addressability to the nanostructure.

In some embodiments, the architecture is made from a single stranded oligomer, as described in U.S. Pub. No. 2018/0044372 and herein incorporated by reference. A single strand of DNA used for assembling a nanostructure in accordance with the present disclosure may vary in length. In some embodiments, a single strand of DNA has a length of 500 nucleotides to 10,000 nucleotides, or more. For example, a single strand of DNA may have a length of 500 to 9000 nucleotides, 500 to 8000 nucleotides, 500 to 7000 nucleotides, 500 to 6000 nucleotides, 500 to 5000 nucleotides, 500 to 4000 nucleotides, 500 to 3000 nucleotides, 500 to 2000 nucleotides, 500 to 1000 nucleotides, 1000 to 10000 nucleotides, 1000 to 9000 nucleotides, 1000 to 8000 nucleotides, 1000 to 7000 nucleotides, 1000 to 6000 nucleotides, 1000 to 5000 nucleotides, 1000 to 4000 nucleotides, 1000 to 3000 nucleotides, 1000 to 2000 nucleotides, 2000 to 10000 nucleotides, 2000 to 9000 nucleotides, 2000 to 8000 nucleotides, 2000 to 7000 nucleotides, 2000 to 6000 nucleotides, 2000 to 5000 nucleotides, 2000 to 4000 nucleotides, or 2000 to 3000 nucleotides. In some embodiments, a single strand of DNA may have a length of at least 2000 nucleotides, at least 3000 nucleotides, at least 4000 nucleotides, or at least 5000 nucleotides. In some embodiments, a single strand of DNA may have a length of 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3100, 3200, 3300, 3400, 3500, 3600, 3700, 3800, 3900, 4100, 4200, 4300, 4400, 4500, 4600, 4700, 4800, 4900, 5100, 5200, 5300, 5400, 5500, 5600, 5700, 5800, 5900, 6600, 6200, 6300, 6400, 6500, 6600, 6700, 6800, 6900, 7100, 7200, 7300, 7400, 7500, 7600, 7700, 7800, 7900, 8100, 8200, 8300, 8400, 8500, 8600, 8700, 8800, 8900, 9100, 9200, 9300, 9400, 9500, 9600, 9700, 9800, 9900, or 10000 nucleotides.

In some embodiments, the architecture is two-dimensional and comprises a single layer of bricks or a single scaffold. The single layer of bricks may form a molecular canvas. In other embodiments, the architecture is three-dimensional and may contain 2, 3, 4, 5, 6, 7, 8, 9, 10, 20, 30, or more layers of two-dimensional structures depending on the desired final shape.

In some embodiments, the architecture is attached to a substrate, such as a glass slide, a silicon base, or a breadboard.

In other embodiments, the architecture remains in a solution. By altering aspects of the solution, such as but not limited to pH, salt concentrations, and cation charge, the aggregation of the bricks may be changed, which may change the orientation, as well as the absorbance spectra, of the chromophores.

Preferred embodiments include, but are not limited to, nucleotide nanostructures, nucleotide particles, nucleotide polypods, and nucleotide hydrogels.

### Dyes

Any dye comprising at least one chromophore in which an exciton is created when excited is acceptable and may be used in any embodiment. A dye may be symmetrical or asymmetrical and may have additional modifications to change solubility, hydrophobicity, or symmetry in order to adjust the placement of the dye (i.e., its proximity and orientation to another dye or aggregate). By way of non-limiting examples, the dye may be one or more of a xanthene derivatives such as fluorescein, rhodamine, Oregon green, eosin, and Texas red; cyanine derivatives such as cyanine, indocarbocyanine, oxacarbocyanine, thiacarbocyanine, and merocyanine; a squaraine derivative or ring-substituted squaraines such as Seta, SeTau, and Square dyes; a naphthalene derivative such as a dansyl or prodan derivative; a coumarin derivative; a oxadiazole derivative such as pyridyloxazole, nitrobenzoxadiazole and benzoxadiazole; an anthracene derivatives such as anthraquinones including DRAQS, DRAQ7 and CyTRAK Orange; a pyrene derivative such as cascade blue; an oxazine derivative such as Nile red, Nile blue, cresyl violet, oxazine 170; an acridine derivative such as proflavin, acridine orange, acridine yellow; and an arylmethine derivative such as auramine, crystal violet, and malachite green; a tetrapyrrole derivative such as porphyrins, chlorin, porphin, phthalocyanine, and bilirubin; or a dipyrromethene derivative, such as, but not limited to, a BODIPY family dye which have the general formula of C9H7BN2F2, for example 4,4-difluoro-4-bora-3a,4a-diaza-s-indacene. The aggregates may alternatively comprise one or more commercial dye(s), such as but not limited to Freedom™ Dye, Alexa Fluor® Dye, LI-COR IRDyes®, ATTO™ Dyes, Rhodamine Dyes, or WellRED Dyes; or any other dye. Examples of Freedom™ Dyes include 6-FAM, 6-FAM (Fluorescein), Fluorescein dT, Cy3™, TAMRA™, JOE, Cy5™, TAMRA, MAX, TET™, Cy5.5™, ROX, TYE™ 563, Yakima Yellow®, HEX, TEX 615, TYE™ 665, TYE 705, and Dyomic Dyes. Examples of Alexa Fluor® Dyes include Alexa Fluor® 488, 532, 546, 647, 660, and 750. Examples of LI-COR IRDyes® include 5′ IRDye® 700, 800, and 800CW. Examples of ATTO™ Dyes include ATTO™ 488, 532, 550, 565, Rho101, 590, 633, 647N. Examples of Rhodamine Dyes include Rhodamine Green™-X, Rhodamine Red™-X, and 5-TAMRA™. Examples of WellRED Dyes include WellRED D4, D3, and D2. Examples of Dyomic Dyes include Dy-530, -547, -547P1, -548, -549, -549P1, -550, -554, -555, -556, -560, -590, -591, -594, -605, -610, -615, -630, -631, -632, -633, -634, -635, -636, -647, -647P1, -648, -648P1, -649, -649P1, -650, -651, -652, -654, -675, -676, -677, -678, -679P1, -680, -681, -682, -700, -701, -703, -704, -705, 730, -731, -732, -734, -749, -749P1, -750, -751, -752, 754, -756, -757, -758, -780, -781, -782, -800, -831, -480XL, -481XL, -485XL, -510XL, -511XL, -520XL, -521XL, -601XL. Examples of other dyes include squaraine, 6-FAM, Fluorescein, Texas Red®-X, and Lightcycler® 640.

In some embodiments, the dyes are bound to the 5′ ends of the nucleotide bricks. In other embodiments, the dyes are bound to the 3′ ends of the nucleotide bricks. In yet other embodiments, the dyes are bound internally within the nucleotide bricks. In still more embodiments, the dyes are bound to any mix of 5′ ends, 3′ ends, or internally. The position of the chromophore within the dye will depend on the desired final configuration. Methods of binding dyes to nucleotides are well known in the art.

In some embodiments, the dyes are bound to the same nucleotide duplex. In other embodiments, the dyes are bound to separate nucleotide duplexes.

In some embodiments, some of the dyes are covalently bound to the bricks while other of the dyes are bound to separate oligomers, which is a linker nucleotide oligomer, and the linker oligomers then Watson-Crick pair with exposed single strands of the bricks.

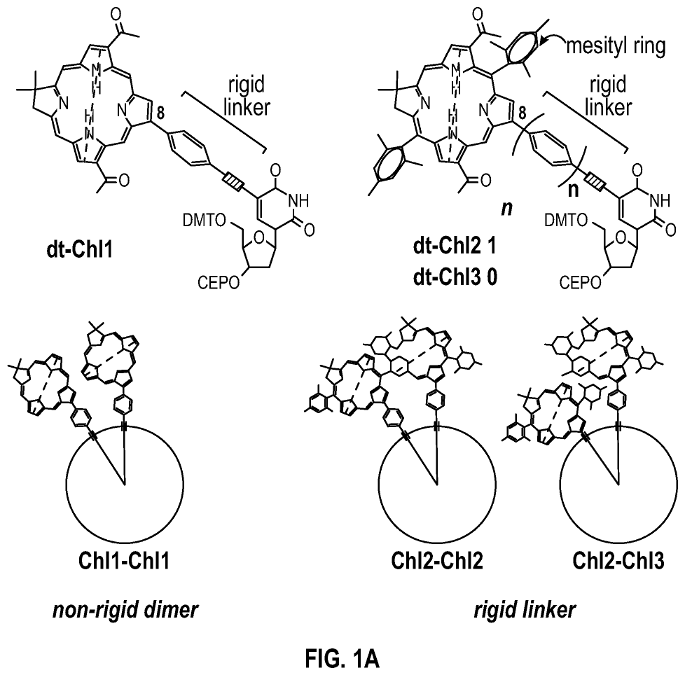

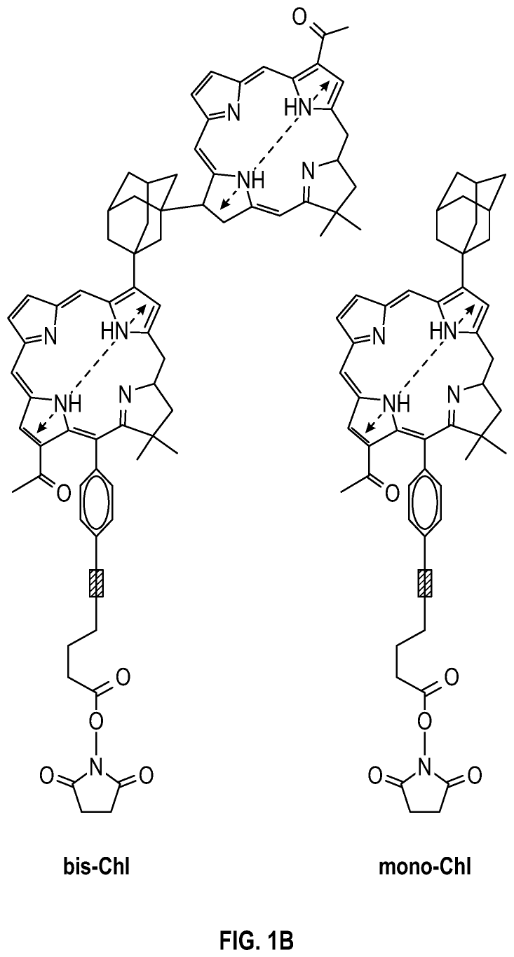

Dyes, commercially available, including custom dyes, or dyes produced in-house may also be modified to control their steric hindrance or their symmetry, as described in U.S. application Ser. No. 16/739,963 (herein incorporated in its entirety) or by substituting the dye with a steric factor which will turn the dye relative to its core (FIG. 1A). For example, by substituting a ring structure like rotaxane around the dyes their steric hindrance may be increased to prevent H-like aggregation or to hold them in a more desired configuration by inhibiting torsional rotation. The ring structure may be further substituted to increase steric hindrance. Further, the number of rings may be increased to increase steric hindrance. As another example, a methylated benzene ring, such as 1,3,5-trimethylbenzene (mesitylene) may be substituted onto the dye. The methyl groups may act to inhibit the dyes torsional rotation. Restricting torsional rotation or increasing steric hinderance may promote the desired orientation between two or more dyes. For example, the mesitylene groups may be positioned so that two dyes may be held in place with J-like stacking.

### Dye Aggregates

When two or more dyes are placed sufficiently close to each other to couple, they may form an aggregate. The stronger the coupling of the dyes within the aggregate, the greater the peak shift relative to the monomer or the greater the Davydov splitting will be. Aggregates have different properties than the chromophores that make up the aggregate, as can be seen in the differences between the Hamiltonians between an aggregate and a single chromophore.

The aggregate based excitonic quantum electronic coherence effects may occur at room temperature in wet and noisy environments and the systems may be less than about 10 nm in extent. These systems provide several large benefits over the currently available excitonic quantum coherent systems, which are much larger, measuring in the micrometer size, and require extreme operating conditions, such as cryogenic temperatures, external magnetic fields and/or large microwave pulses, and dry environments.

When dyes aggregate in high concentrations in solution, coherent exciton delocalization behavior (e.g., large Davydov splitting, exchange narrowing, circular dichroism, Cotton effects, or Stokes shifting) can be observed. The circular dichroism may be either right-or left-handed and so may absorb light having particular circular polarization.

Using the above architectures, one or more dyes, each comprising at least one chromophore or fluorophore, may be placed in precise distances from each other to create different coupling strengths of their dipoles unlike relying on high aggregation concentration in a solution. At sufficiently short distances on these architectures, the dipoles of two or more chromophores residing on one or more dyes may become weakly to strongly coupled and may form an aggregate for exciton sharing. Alternatively, the dipoles may be positioned to allow very weak coupling for FRET.

In some embodiments, using the above architectures, two or more dyes may be bound to the nucleotides in order to be precisely placed so that the chromophores create weak to strongly coupled aggregates. When so placed, the aggregates may produce quantum electronic coherent excitons, biexcitons, triexcitons, or higher excitons, when excited by a light source. In some exemplary embodiments, the two or more dyes are covalently bound to the same nucleotides brick, and then the dye-bound brick and any non-bound bricks are allowed to self-assemble into the desired final one-, two-, or three-dimensional shape. In another embodiment, the two or more dyes are covalently bound to different nucleotide bricks and then the bricks are allowed to self-assemble into the desired final one-, two-, or three-dimensional shape. Another embodiment is combination thereof, in which some bricks have one bound dye while other bricks have multiple bound dyes. The bricks once assembled place the dyes within their aggregate.

In some embodiment, the bricks, which may already have bound dyes, are allowed to first self-assemble into the desired final one-, two-, or three-dimensional shape. Portions of the bricks may still be unpaired after assembly, allowing for further binding of complementary oligomers. The two or more dyes are bound to at least one complementary oligomer which may then pair with the one or more unpaired portions of the bricks.

The orientation of the two or more chromophore transition dipole moments, occurring within the dyes, to each other may affect the absorbance and emission spectra of the aggregate. Depending on the orientation, a pair of chromophores (dimer) sufficiently close to allow weak to strong excitonic coupling will have different characteristics when compared to the monomer chromophore. When the transition dipole moments are more or less parallel in the dimer an “H-dimer” forms that shows H-like stacking, or H-like coupling, which is characterized by a blue-shift in absorbance due to having a higher excited energy state when compared to the monomer. When the transition dipole moments are more or less in an end-to-end orientation in a dimer, a “J-dimer” forms that shows J-like stacking, or J-like coupling, which is characterized by a red-shift in absorbance due to having a lower excited energy state when compared to a monomer. Real systems exhibit some deviation from the ideal face-to-face or end-to-end orientations of transition dipole moments that can be described with dimer obliquity. When the transition dipole moments of a dimer are at about 90 degrees, the dimer is defined as a purely or (ideally) oblique dimer, in which an equally mixed “J/H-dimer” form. Here, Davydov splitting is observed in the absorbance spectrum because both a higher and lower excited energy state are allowed by selection rules when compared to a monomer (see Cannon et al., Coherent Exciton Delocalization in a Two-State DNA-Templated Dye Aggregate System, 2017, J. Phys. Chem. A, 121: 6905-6916, herein incorporated by reference). For those dimers that are not purely J-like or H-like, nor purely oblique, both energy states are allowed to different degrees dependent on the angle between them. When weakly allowed, the states may not be optically observable in the absorbance spectrum.

As taught in U.S. patent application Ser. No. 16/100,052 (herein incorporated by reference in its entirety), the orientation of the chromophores within an aggregate on a linear oligomer, is also affected by characteristics of the solution, including salt concentration, temperature, and cation concentration. The orientation may affect the absorbance spectra. Therefore, by altering the conditions of the solution, it is possible to fine tune the absorbance spectra of the aggregates. For example, as the salt concentration increases, a dimer aggregate may be fine-tuned to exhibit either J-dimer characteristics at lower salt concentrations or H-dimer characteristics at high salt concentrations. Also, altering both the temperature and salt concentrations, it is further possible to tune the chromophores for specific characteristics as not only the absorbance, but the emission may be altered by changing the concentration of salt in the solution.

Additionally, the absorbance spectrum of the aggregates on a linear oligomer is also affected by the spacing of the chromophores comprising the aggregate. As the distance increases between the chromophores, coupling is decreased which leads to diminished spectral absorbance shifts from that of the monomeric dyes and the absorbance spectrum will approach that of the monomer. In order for strong coupling to occur in dye aggregates, dyes must be closely spaced, but to prevent two aggregates behaving as a single aggregate, an aggregate must be spaced so that they are only moderately or weakly coupled with another aggregate.

Taken together, by altering the composition of the solution surrounding the nucleotide architecture and by altering the distance between the chromophores and aggregates, one skilled in the art may alter the absorbance and emission spectra for an aggregate comprised of two or more chromophores bound to a nucleotide architecture.

In a preferred embodiment, an aggregate comprises at least two chromophores held by a rigid linker to a nucleotide architecture so that the transition dipole moments are arranged in an end-to-end orientation, such as a head-to-tail or head-to-head orientation, to form a J-dimer. In another preferred embodiment, three chromophores are held in a head-to-tail or head-to-head orientation to form a triangular J-trimer.

Other embodiments comprising J-dimers may also be used. In another embodiment, the aggregate comprises at least two chromophores held oblique to each other by an architecture to form a mixed J/H-dimer.

In another embodiment, an aggregate comprises at least three chromophores are positioned within the nucleotide architecture so that two of the three chromophores form a J-dimer, and two of the three chromophores form a H-dimer. In a different embodiment, an aggregate comprises chromophores positioned such that two form a J-dimer and the third forms two mixed J/H-dimers. In yet another embodiment, an aggregate comprises three chromophores with two chromophores forming a H-dimer and the third forms two mixed J/H-dimers.

In yet another embodiment, an aggregate comprises a tetramer of chromophores positioned within the architecture such that two H-dimers, two J-dimers are formed, and two mixed J/H-dimers form. In other embodiments, the tetramer can be position so that two H-dimers and four mixed J/H-dimers are formed. In yet another embodiment, the tetramer is positioned so that two J-dimers and four mixed J/H-dimers are formed.

In some embodiments, an aggregate comprises chromophores which all have the same optical transition energies. In other embodiments, the chromophores differ in their optical transition energies. The different optical transition energies allow the construction of a set phase shifter having desired values of absorbance and emittance.

In other embodiments, the basic configurations dimer, trimers, and tetramers as described above can be joined with other monomers, dimer, trimers, and tetramers in order to form more complex aggregates. An aggregate may comprise of any number of dyes, or chromophores, for example an aggregate may comprise of 2 or more, 3 or more, 4 or more, 5 or more, 7 or more, 10 or more, or 15 or more dyes. Preferably an aggregate may comprise from between 2 and 20 chromophores, from between 2 and 15 chromophores, from between 2 and 10 chromophores, or from between 2 and 5 chromophores. In a most preferred embodiment, the aggregate comprises of a dimer or trimer of chromophores.

### Linkers Between the Dye and Nucleotide

In order to gain access to chromophores having J-like stacking of the transition dipole moments as a source of fluorescent material, the dye may be rigidly linked to a substrate. By using a rigid linker, the radiative rate, kᵣ, may be improved in multiple ways. For example, by holding the chromophores in a specific orientation, access to the lower excited electronic state, the red shift, due to the J-like stacking of the chromophores of a multimer aggregate when compared to the monomer may be obtained, resulting in a narrowing between the absorption and emission peaks. This narrowing improves the radiative rate by bringing the emission peak closer to the absorbance peak so that the two states are in near resonance thereby increasing the radiative rate, kᵣ. See M. Gross and S. Haroche, Superradiance: An essay on the theory of collective spontaneous emission, Physical Reports, 93 (5), 301-396 (1982); R. H. Dicke, Coherence in spontaneous radiation processes, Physical Review, 93 (1), 99-110 (1954); and R. Bonifacio and L. A. Lugiato, Cooperative radiation processes in two-level systems: Superfluorescence, Physical Review A, 11 (5), 1507-1521 (1975), each herein incorporated in their entirety.

The non-radiative rate will also be reduced by limiting the non-radiative transitions of the dye. The rigid linker may reduce the amount of vibrational freedom of the dye, limiting its interactions with surrounding molecules, such as water or nucleotides. As there are fewer vibrational pathways to lose energy, the excitation may live longer, increasing the dephasing time, τ_{d}, and allowing for a greater probability of the excitation to relax back to the ground state and emit visible light instead of increasing the heat of the system or producing infrared light.

Additionally, increasing the rigidity, by reducing torsional rotation, may also increase the dephasing time, τ_{d}, which may mitigate excited state quenching and improve kᵣ.

In preferred embodiments, the substrate is a nucleic acid, nucleoside, or nucleotide. The substrate may also be an oligonucleotide or a nucleotide architecture.

The linker may be any rigid molecule or compound that will limit the movement of the dye to promote J-like, or end-to-end, stacking of the transition dipole moment (μ) of the dye. The length of the linker may be varied depending on the selected dye(s) to allow for dye specific tuning of the chromophores.

In preferred embodiments, the linker is a cyclic or acyclic C₂ to C₃₀ alkyne, more preferably a cyclic or acyclic C₂ to C₂₀ alkyne, and even more preferably a cyclic or acyclic C₂ to C₈ alkyne. In a particularly preferred embodiment, the linker is acetylene.

In various embodiments, the linker may be substituted, and the dye may be attached to the substituted compound (FIG. 1A). A preferred substitute is substituted or unsubstituted benzene. Benzene is a preferred substitute due to its stability, rigidity, and size. To lengthen the linker, longer alkynes may be used. Optionally, multiple units of a substitute, such as benzene may be used. The number of units may be from about 1 to about 20, more preferably from about 1 to about 10, and most preferably from about 1 to about 5.

### Bridges

Two or more dyes may also be covalently bridged in a non-conjugating fashion to form a single molecule. The structure of the bridge is critical and should meet the following requirements: (1) the bridge should be rigid to prevent the chromophores' conformational rotation; (2) does not form a conjugated system with the chromophores; (3) should not be too short to avoid mixing of the chromophores' ground states; and (4) should not be too long to allow the transition dipole moments to couple.

Similar to the linkers, these bridges may increase the rigidity between dyes to increase the dephasing time, τ_{d}, and decrease orbital overlap, which may mitigate excited state quenching and improve kᵣ.

Further, due to the bridge combining the dyes into one molecule while maintaining the positions of the dyes, and hence their transition dipole moments, in end-to-end orientation, the molecule may have two or more transition dipole moments (FIG. 1B).

Preferred bridges are carbon cages and bicyclic bridges, such as, but not limited to, diamondoids. Diamondoids may be used for dyes that do not have optical absorption deep in the ultraviolet spectral region as they have optical absorption in that spectrum with optical band gaps around 6 electron-volts (eVs) and higher. Examples of diamondoids include, but are not limited to, adamantane, iceane, BC-8, diamantine (diadamantane), triamantane (triadmantane), isotetramantane, pentamantane, cyclohexamantane, and super-adamantane.

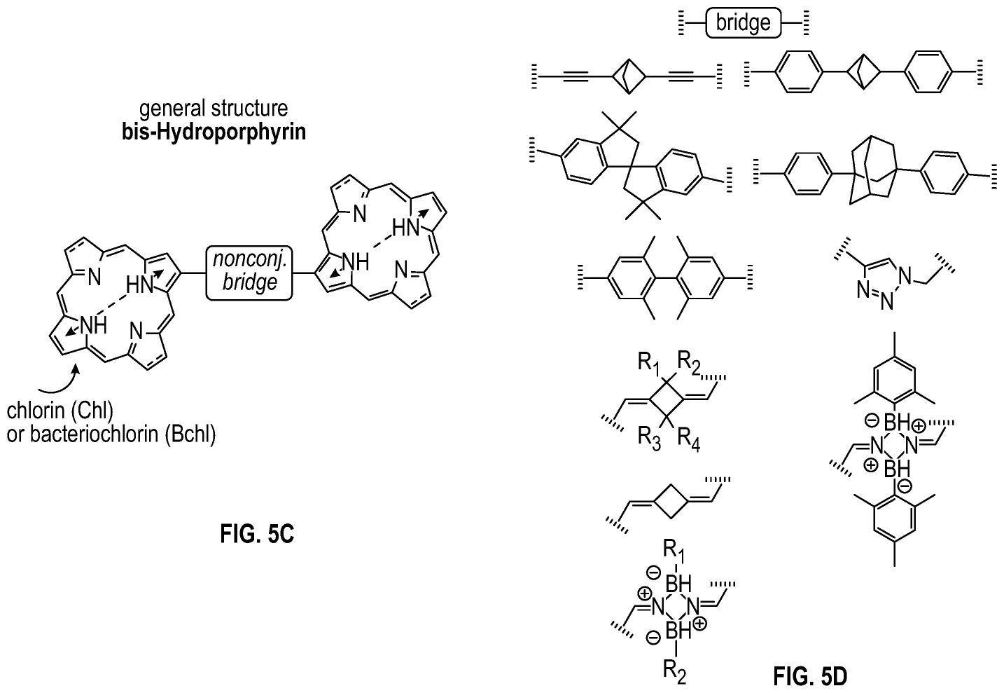

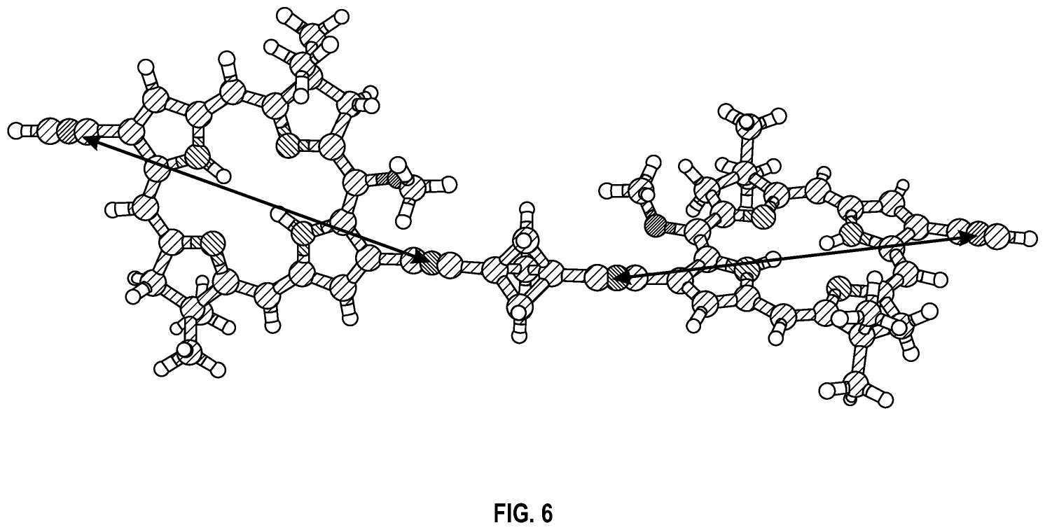

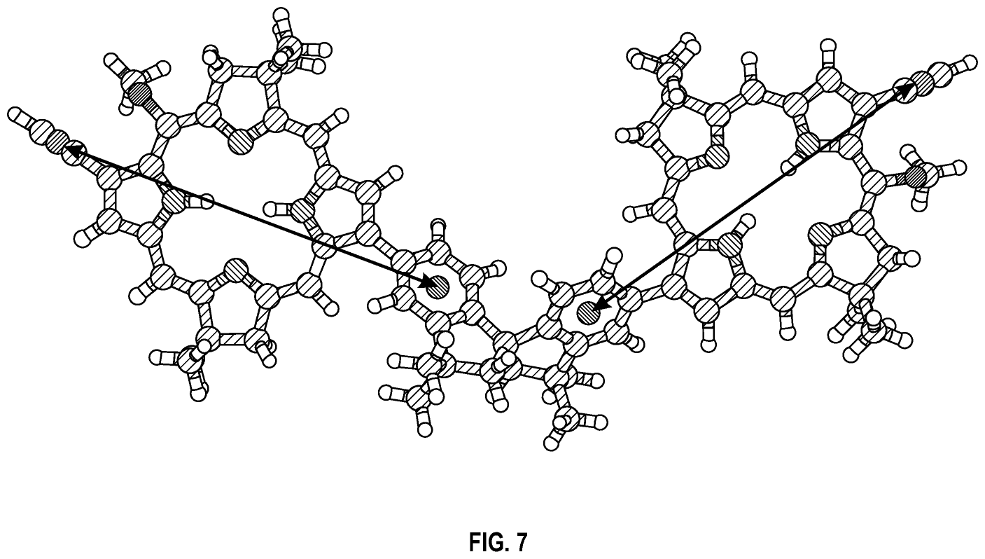

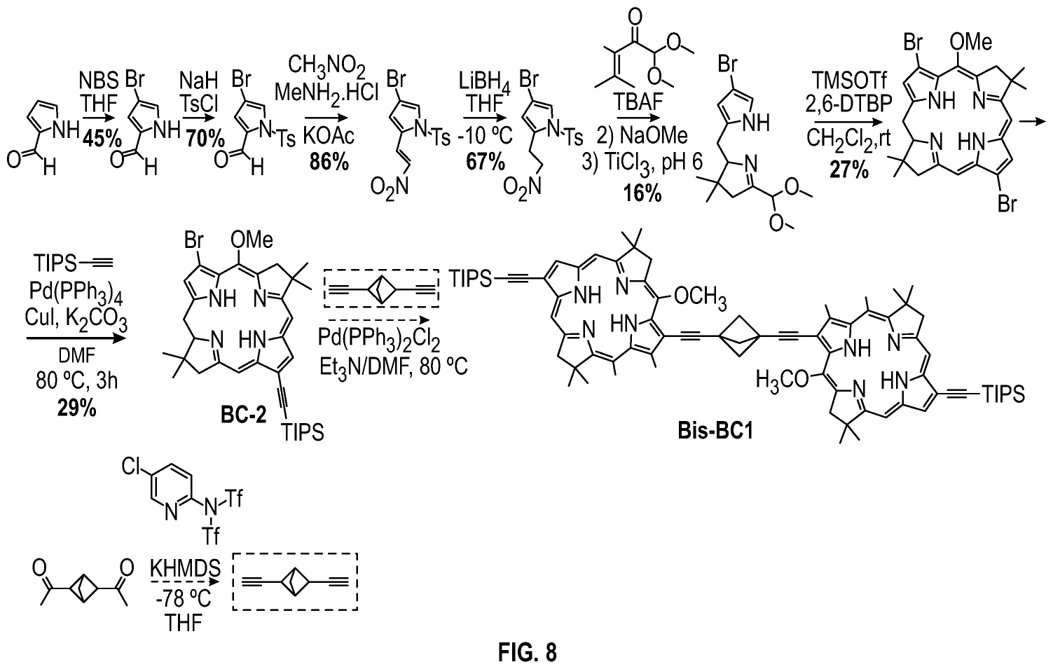

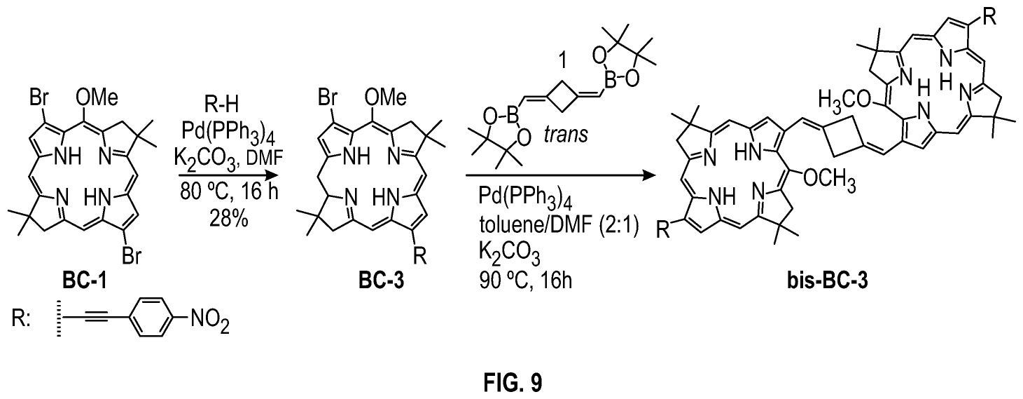

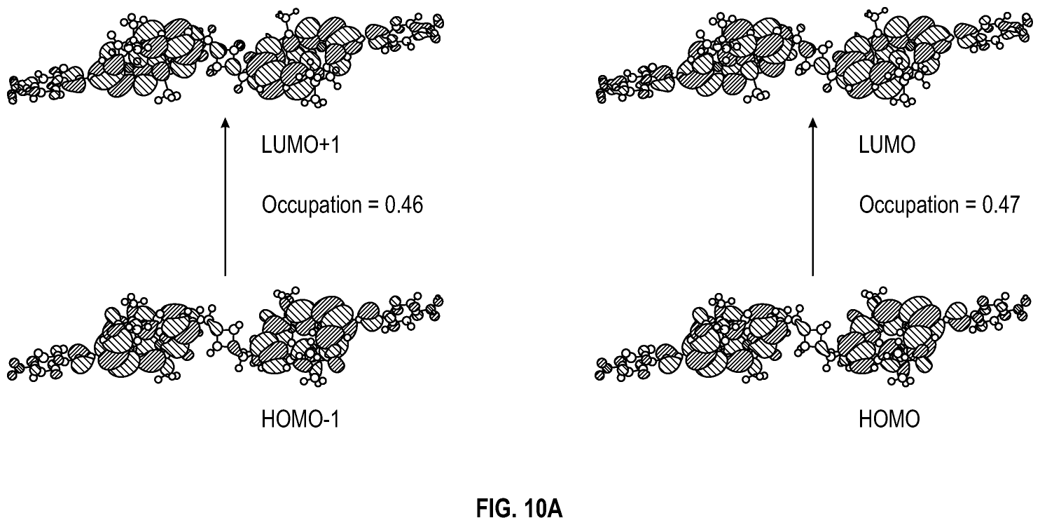

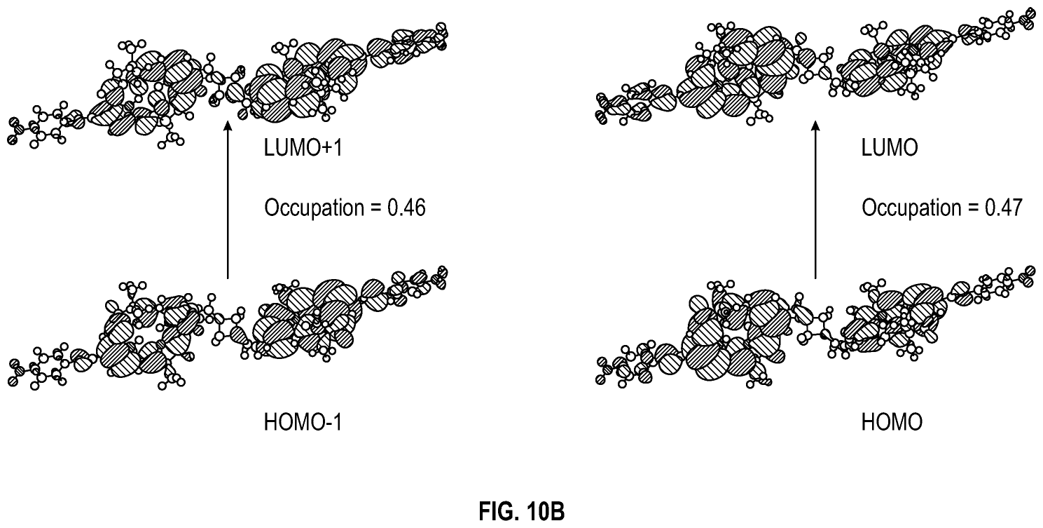

Two specific chemical structures of non-conjugating rigid bridges, cyclo[1.1.1]pentane-type bridge and spiro[4.4]nonane-type bridge (FIG. 5), can be used in lieu of or in addition to the previously identified adamantane bridge. In the proposed designs, the custom, in-house made bacteriochlorin dyes are attached to those bridges to provide a J-packing with a 2.0-4.0 Å end-to-end distance between long wavelength (Q_{y}) transition dipole moments (μ). The acetylene functional group (protected with TIPS) is intended to be used to attach a linker for tethering to DNA or to attach the third chromophore.

Preferred bicyclic bridges include fused and bridged bicyclic compounds. More preferrable bicyclic bridges include, but are not limited to, bicyclopentane, bicyclooctane, triptycene, or cubane. It is preferable that the bicyclic compound is not spirocyclic.

Other bridges may include cyclic or acyclic C₂ to C₃₀ alkylene, alkene, or alkyne, more preferably cyclic or acyclic C₂ to C₂₀ alkylene, alkene, or alkyne, and even more preferably cyclic or acyclic C₂ to C₈ alkylene, alkene, or alkyne. The bridge may be further substituted to meet one of the four requirements for a bridge.

A bridged dye aggregate may comprise of any number of dyes covalently bonded through one or more bridges. A bridged dye system may have from about 1 to about 10,000 dyes, from about 1 to about 1,000 dyes, or from about 1 to about 100 dyes. In preferrable embodiments, each dye is separated from another dye by a bridge. However, multiple dyes may be attached to the same bridge.

### Frenkel Molecular Excitation Theory and Corresponding Key Parameters

The physical parameters J and K (defined below) can be tuned using DNA-templated dye networks, which forms a system of Frenkel (molecular) excitons. The details and significance of these physical parameters are discussed in the following. Insight to these physical parameters is best gleaned by defining the following aspects of the Frenkel Model, which describes a system of Frenkel (molecular) excitons: (1) the single-molecule energy eigenstates, (2) the multi-exciton Hilbert space, and (3) the Frenkel Hamiltonian governing the system.

### Single-Molecule Energy Eigenstates

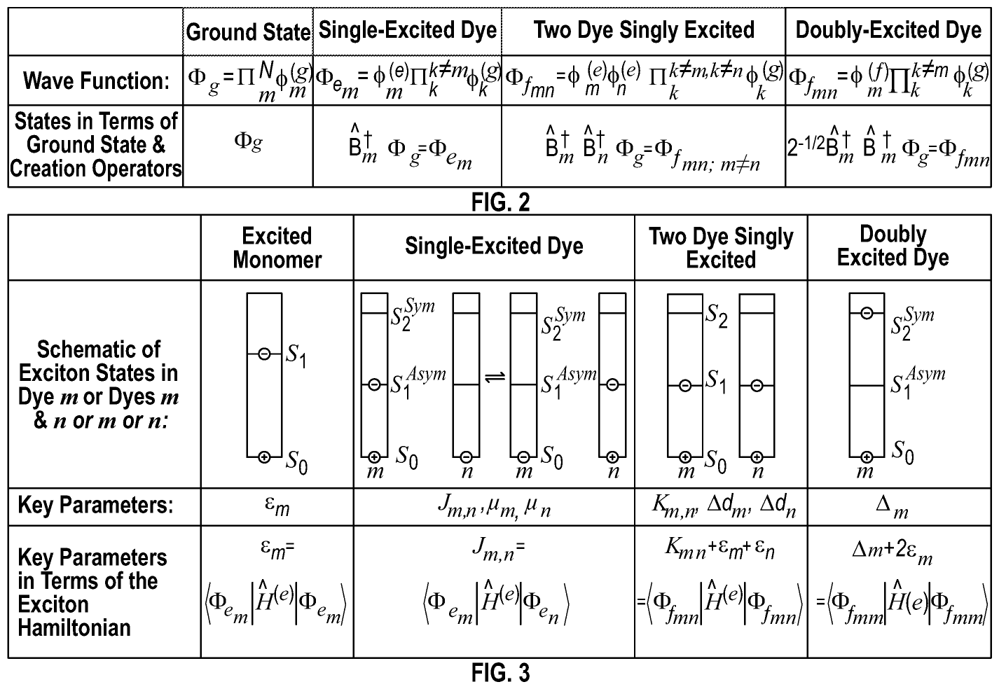

By way of a nonlimiting example, consider an aggregate comprised of N three-level dyes in which only interactions between pairs of dyes are considered (i.e., between the dye at site m and the dye at site n). The single dye energy eigenstates are represented by three wave functions (Table 1): (1) the ground-state wave function here taken to be a singlet state, ϕₘ^{(g)}, (2) the singly excited-state wave function, here taken to be the lowest singlet state, ϕₘ^{(e)}, and (3) the double-excited state wave function, here taken to be the next higher singlet state, ϕₘ^{(f)}, where m denotes the dye at site m. Invoking the Heitler-London approximation the ground state of the aggregate is given by a direct product of the ground states of all dyes, Φ_{g}, and the single-exciton basis is constructed by replacing one of the dye ground states by its excited state and is given by Φₑ_{ₘ}. The wave functions are shown in FIG. 2.

### Multi-Exciton Hilbert Space

Note that exciton states that exhibit exciton delocalization are created by the interaction of two (or more) strongly coupled dyes resulting in excited states that are split (i.e., Davydov splitting) as compared to the monomer state (see schematics in Table 2). Focusing on dye dimer aggregates, the exciton states can be described by two-exciton basis states that come in two forms: dyes (m) that are doubly excited Φ_{f}_{ₘₘ}), and two dyes (m&n) that are singly excited (Φ_{f}_{ₘₙ}) which are shown in Table 1. This model contains N singly excited states and M=N(N+1)/2 doubly-excited states. An approximation is often made that excludes the states where a dye molecule is doubly excited. This is the hard-core boson approximation (i.e., two excitons cannot reside on the same dye). Inclusion of these states, as done here, constitutes the soft-boson approximation (i.e., two excitons can reside on the same dye). Schematics of the exciton energy states are shown in FIG. 3.

### Frenkel Hamiltonian Governing a System of Frenkel (Molecular) Excitons

The behavior of Frenkel excitons is well approximated by an augmented Frenkel Hamiltonian that includes (see Table 2) exciton exchange energies, J_{m,n} (i.e., excitonic hopping parameter; a single exciton non-permanent dipole-dipole coupling between dyes on sites m and n), leading to resonant exciton hopping; and the exciton-exciton interaction energies, (permanent dipole-dipole coupling between two excitons, one each on dye sites m and n). Both J_{m,n} and K_{m,n}, are key parameters that we propose that can be harnessed to create, study, and control exciton quantum entanglement. An augmented Frenkel Hamiltonian of this form is given by:

$${\hat{H}}^{( e )} = {\sum }_{m} {ε}_{m}^{e} {\hat{B}}_{m}^{†} {\hat{B}}_{m} + {\sum }_{m , n}^{m \neq n} {J}_{m , n} {\hat{B}}_{m}^{†} {\hat{B}}_{n} + \frac{1}{2} {\sum }_{m , n}^{m \neq n} {K}_{m , n} {\hat{B}}_{m}^{†} {\hat{B}}_{n}^{†} {\hat{B}}_{m} {\hat{B}}_{n} + \frac{1}{2} {\sum }_{m} {Δ}_{m} {\hat{B}}_{m}^{†} {\hat{B}}_{m}^{†} {\hat{B}}_{m} {\hat{B}}_{m}$$

where εₘ^{e} is the monomer transition energy of a single excited dye (excited monomer: So→S1) on the m site, \hat{B}ₘ^{†}is the bosonic exciton creation operator on site m, and \hat{B}ₘ is the bosonic exciton annihilation operator on site m. K_{m,n} is related to the average energy of two singly excited dyes (see FIG. 3), given by:

ε_{f,mn}^{e}= [symbol] Φ_{f}_{ₘₙ}|Ĥ^{(e)}|Φ_{f}_{ₘₙ} [symbol] =εₘ^{e}+εₙ^{e}+Kₘₙ

Similarly, Δₘ is related to the average energy of a doubly excited dye:

ε_{f,mm}^{e}= [symbol] Φ_{f}_{ₘₘ}|Ĥ^{(e)}|Φ_{f}_{ₘₘ} [symbol] =2εₘ^{e}+Δₘ

This equation does not describe three exciton states or higher.

### Dipole Approximation and Define J_{m,n}, K_{m,n} and Δd

When intermolecular distances are greater than dye size, the dipole approximation for molecular charge densities can be further invoked such that the J_{m,n} and K_{m,n} couplings can be expressed in dipole-dipole interaction form. J_{m,n} becomes the intermolecular dipole-dipole interaction between the molecular transition dipoles, μₘ and μₙ, for the dyes at sites m and n, which is given by:

$${J}_{m , n} = \frac{1}{4 π ε {ε}_{o}} ( \frac{{μ}_{m} \cdot {μ}_{n}}{{\vert {R}_{m , n} \vert}^{3}} - 3 \frac{( {μ}_{m} \cdot {R}_{m , n} ) ( {μ}_{n} \cdot {R}_{m , n} )}{{\vert {R}_{m , n} \vert}^{5}} )$$

where R_{m,n} is the vector connecting dyes at sites m and n. K_{m,n} involves the difference between the excited state and ground state static (i.e., permanent) dipoles, Δdₘ and Δdₙ, also known as the difference static dipole moments, for the dyes at site m and n, and is given by:

$${K}_{m , n} - \frac{1}{4 π ε {ε}_{o}} ( \frac{Δ {d}_{m} \cdot Δ {d}_{n}}{{\vert {R}_{m , n} \vert}^{3}} - 3 \frac{( Δ {d}_{m} \cdot {R}_{m , n} ) ( Δ {d}_{n} \cdot {R}_{m , n} )}{{\vert {R}_{m , n} \vert}^{5}} )$$

and thus K_{m,n}, is zero for a molecule without a difference static dipole moment (e.g., a symmetric dye, Δd=0). Although Eqns. 4 and 5 provide straightforward expressions to give an indication of the parameters on which they depend, for intermolecular distances that are less than the dye size (i.e., the dipole-dipole approximation fails), more accurate expressions for J_{m,n} and K_{m,n} are necessary and are used in the KRM Model Simulation Tool (see B. L. Cannon, D. L. Kellis, L. K. Patten, P. H. Davis, J. Lee, E. Graugnard, B. Yurke and W. B. Knowlton, Coherent exciton delocalization in a two-state DNA-templated dye aggregate system, The Journal of Physical Chemistry A, 121 (37), 6905-6916 (2017) and B. L. Cannon, L. K. Patten, D. L. Kellis, P. H. Davis, J. Lee, E. Graugnard, B. Yurke and W. B. Knowlton, Large Davydov splitting and strong fluorescence suppression: An investigation of exciton delocalization in DNA-templated Holliday junction dye aggregates, The Journal of Physical Chemistry A, 122 (8), 2086-2095 (2018), herein incorporated by reference in their entirety) based on the Kühn-Renger-May (KRM) model (O. Kühn, T. Renger, and V. May, Theory of exciton-vibrational dynamics in molecular dimers, Chemical Physics, 204 (1), 99-114 (1996)) used to fit absorbance and circular dichroism data. Density functional theory (DFT) and TD-DFT also produces more accurate expressions for J_{m,n} and K_{m,n}.

### The Hamiltonian for Vibrons

The augmented Frenkel Hamiltonian expression (Eqn. 1) does not include the coupling effects of the vibronic quanta (i.e., vibrons) to the excitons of the system. The Hamiltonian, a Holstein-like Hamiltonian, for the vibrons and their coupling to the excitons can be expressed as:

$${\hat{H}}^{( v )} = {\sum }_{m} {\sum }_{α} {ε}_{m , α}^{v} {\hat{A}}_{m , α}^{†} {\hat{A}}_{m , α} + {\sum }_{m} {\sum }_{α} {D}_{m , α} {\hat{B}}_{m}^{†} {\hat{B}}_{m} ( {\hat{A}}_{m , α}^{†} + {\hat{A}}_{m , α} )$$

where a Â_{m,α}^{†} is the vibron creation operator, Â_{m,α}is the vibron annihilation operator, is the corresponding vibron energy, and D_{m,α}is the displacement parameter between the electronic ground state and the electronic excited-state harmonic oscillator potentials — all for vibronic mode a on the dye at site m. The meaning of ε_{m,α}^{v} and ε_{m,α}^{v} is illustrated schematically in FIG. 2 where ε_{m,α}^{v} is the difference in energy between two neighboring vibronic energy states while D_{m,α}is the difference between the electronic ground and excited state minimums. The sum of Eqns. 1 and 6 gives an augmented Frenkel Hamiltonian, or Frenkel-Holstein Hamiltonian, that includes vibronic effects on excitons and is given by:

$$\begin{matrix}\hat{H} = {\hat{H}}^{( e )} + {\hat{H}}^{( v )} \\ = {\sum }_{m} {ε}_{m}^{e} {\hat{B}}_{m}^{†} {\hat{B}}_{m} + {\sum }_{m , n}^{m \neq n} {J}_{m , n} {\hat{B}}_{m}^{†} {\hat{B}}_{n} + \frac{1}{2} {\sum }_{m , n}^{m \neq n} {K}_{m , n} {\hat{B}}_{m}^{†} {\hat{B}}_{n}^{†} {\hat{B}}_{m} {\hat{B}}_{n} + \\ \frac{1}{2} {\sum }_{m} {Δ}_{m} {\hat{B}}_{m}^{†} {\hat{B}}_{m}^{†} {\hat{B}}_{m} {\hat{B}}_{m} + {\sum }_{m} {\sum }_{α} {ε}_{m , α}^{v} {\hat{A}}_{m , α}^{†} {\hat{A}}_{m , α} + \\ {\sum }_{m} {\sum }_{α} {D}_{m , α} {\hat{B}}_{m}^{†} {\hat{B}}_{m} ( {\hat{A}}_{m , α}^{†} + {\hat{A}}_{m , α} )\end{matrix}$$

### Key Parameters

Both J_{m,n} and K_{m,n} along with their associated parameters can be used to create and control exciton quantum entanglement and to construct excitonic quantum gates. Hence, the strength of coupling between dyes m and n—in which one dye is singly excited (one exciton), both dyes are excited singly excited (two exciton), or one dye is doubly excited (two excitons) and the orientation of the dyes relative to one another—is critically important to exciton quantum entanglement and quantum computing. Super-resolution microscopy and spectroscopy, atomic force microscopy (AFM) and metrology of DNA origami, which may be used to quantify the precision with which dye orientation can be controlled using DNA.

From Eqn. 4, we see that J_{m,n} is related to the transition dipole moments, μₘ and μₙ, of dyes m an n, which are parallel to the incident electromagnetic (EM) field inducing and oscillating the dipoles. The square of a single dye's transition dipole, μₘ², is proportional to the extinction coefficient. These relationships indicate that dyes with larger extinction coefficients will yield aggregates with larger J_{m,n}. J_{m,n} can be determined from the amount of Davydov splitting (˜2J_{m,n} for dimers) obtained from steady state absorption and by using the KRM Model Simulation Tool.

From Eqn. 5, K_{m,n} is proportional to the change in excited-and ground-state difference static (permanent) dipole moments, Δdₘ and Δdₙ of dyes m and n. K_{m,n} can be determined using ultrafast nonlinear spectroscopy, while Δdₘ can be obtained using Stark spectroscopy. Another key parameter is the excitonic coherence lifetime τ_{c}. τ_{c} is related to the number of times an exciton can be coherently exchanged between dyes:

n=J_{m,n}τ_{c}c

where c is the speed of light, and J_{m,n} is expressed in units of wavenumber (cm⁻¹). While it can be difficult to measure τ_{c} directly, τ_{c} is related to the absorption spectral width, ΔE, of the optical transitions associated with the excitonic coherence, which can be readily measured via steady state absorption. Spectral narrowing in the form of a reduced ΔE, for example, is an optical signature of exciton delocalization. Such spectral narrowing can be manifested in the form of either motional narrowing (i.e., reduction of homogeneous broadening) or exchange narrowing (i.e., reduction of inhomogeneous broadening). ΔE in the form of homogeneous broadening is related to the intrinsic time scale over which excitonic quantum coherence decays. Ultrafast nonlinear spectroscopy (ensemble construct measurement) and super-resolution imaging (single construct measurement) are needed to disentangle the various homogeneous and inhomogeneous broadening contributions to ΔE and better understand the fundamental mechanisms responsible for the dephasing of the excitonic coherence τ_{c}.

### Mathematical/Theoretical Reasoning

As shown above, J and K are separate and not related. J is dependent on the transition dipole moment and K is dependent on the difference static dipole moment. Additional terms can be added to the Hamiltonian including a term for vibronic effects and for charge transfer.

Chemical Synthesis

¹H NMR spectra (300, 600 MHz) were collected at room temperature in CDCl₃, containing tetramethylsilane as an internal standard. Chemical ¹H shifts (δ) were calibrated using CDCl₃ residual proton signals at 7.26 ppm. Chemical ¹³C shifts (δ) were calibrated using CDCl₃ residual proton signals at 77.2 ppm. All solvents and commercially available reagents were used as received. Compounds BC-1 and 1 were synthesized following the reported procedures.

### 3-bromo-5-methoxy-8,8,18,18-tetramethyl-13-[(4-nitrophenyl)ethynyl]-bacteriochlorin (BC-3)

Following a reported procedure with modifications, a solution of BC-1(26.0 mg, 0.054 mmol), 1-ethynyl-4-nitrobenzene (8.2 mg, 0.056 mmol), and potassium carbonate (74.2 mg, 0.54 mmol) in N,N-dimethylformamide (5.4 mL) in an oven-dried 25-mL Schlenk flask was degassed via three freeze-pump-thaw cycles. Tetrakis(triphenylphosphine)palladium (24.4 mg, 0.022 mmol) was added, and the reaction mixture was stirred at 80° C. for 16 h under argon. The reaction mixture was diluted with ethyl acetate, washed (water and brine), dried (Na₂SO₄), and concentrated using a rotary evaporator. The crude was purified by column chromatography (silica, dichloromethane/hexanes 1:1, then 3:1) to afford BC-3 as a red solid (8 mg, 28%). ¹H NMR (CDCl₃, 600 MHz) δ, ppm: −1.94 (brs, 1H), −1.64 (brs, 1H), 1.95 (s, 6H), 1.96 (s, 6H), 4.35 (s, 3H), 4.43 (s, 2H), 4.44 (s, 2H), 8.00 (d, J=8.7 Hz, 2H), 8.37 (d, J=8.7 Hz, 2H), 8.51 (s, 1H), 8.62 (s, 1H), 8.75 (d, J=2.4 Hz, 1H), 8.85 (d, J=2.0, 1H), 8.93 (s, 1H); λ_{abs} (CH₂Cl₂)/nm 350, 365, 521, 749. ¹³C NMR (CDCl₃, 600 MHz) δ, ppm: 31.1, 31.2, 45.5, 46.3, 48.1, 51.5, 64.9, 90.8, 94.6, 96.47, 96.51, 98.5, 107.2, 114.2, 124.08, 124.10, 124.9, 125.8, 127.7, 130.9, 132.5, 134.8, 135.4, 135.8, 137.9, 147.2, 156.6, 160.9, 169.6, 171.5. HRMS (ESI/Q-TOF) m/z: [M⁺] Calcd for C₃₃H₃₀BrN₅O₃ 623.1532; Found 623.1538.

A solution of BC-3 (7.0 mg, 0.011 mmol), 1 (4.5 mg, 0.013 mmol), and potassium carbonate (15.5 mg, 0.11 mmol) in N,N-dimethylformamide/toluene [0.9 mL, (2:1)] in an oven-dried 25-mL Schlenk flask was degassed via three freeze-pump-thaw cycles. Tetrakis(triphenylphosphine)palladium (2.6 mg, 0.0022 mmol) was added, and the reaction mixture was stirred at 90° C. for 16 h under argon. The reaction mixture was diluted with ethyl acetate/dichloromethane, washed (water and brine), dried (Na₂SO₄), and concentrated using a rotary evaporator. The crude was purified by column chromatography (silica, toluene) to afford bis-BC3 as a red solid.

¹H NMR (CDCl₃, 600 MHz) δ, ppm: −1.82 (brs, 2H), −1.29 (brs, 2H), 1.94 (s, 24H), 3.67 (s, 4H), 4.25 (s, 6H), 4.39 (s, 8H), 7.65 (s, 2H), 8.00 (d, J=8.7 Hz, 4H), 8.37 (d, J=8.7 Hz, 4H), 8.47 (s, 2H), 8.54 (s, 2H), 8.59 (s, 2H), 8.73 (d, 2H), 8.83 (s, 2H); λ_{abs} (toluene) 367, 382, 534, 762 nm; λₑₘ (exc 534 nm, toluene) 771 nm.

### Uses

There are many uses for dyes known in the art. Dyes having J-like or oblique stacking, or coupling, and linked to a nucleotide may be used, by way of nonlimiting examples, as photosensitizers for photodynamic therapy, biosensors, imaging, exciton wires and gates for quantum computing, and as media for optically-pumped nonreciprocal optical devices.

Depending on their use, the nucleotides or oligonucleotides may be further modified. For example, in embodiments where the nucleotides, oligonucleotides, or nucleotide architecture are used as a drug delivery system (see Chi et al. 2020, DNA Nanostructure as an Efficient Drug Delivey Platform for Immunotherapy, Front. Pharmacol., 10:1585; doi:10.3389/fpar.2019.01585 or Hu et al. 2019, DNA nanotechnology-Enabled Drug Delivery Systems, Chem. Rev., 119:6459-6506; doi:10.1021/acs.chemrev.7b00663, both herein incorporated in their entirety) the nucleotides, oligonucleotides, or nucleotide architecture may then further be modified, such as by adding sulfur groups to attach the metal, silicon, or organic microparticles or nanoparticles; or by conjugating a lipid or other hydrophobic molecule for integration into lipid microparticles or nanoparticles. In other embodiments, an oligomer or nucleotide architecture may be modified to have a targeting molecule or signal for cellular or subcellular delivery. In still other embodiments, the nucleotides, oligonucleotides, or nucleotide architecture may be modified to have a signal to aid in cellular uptake, such as through phagocytosis or pinocytosis.

The nucleotides, oligonucleotides, or nucleotide architectures may also comprise of additional components depending on their use. By way of nonlimiting example, a nucleotide-based drug delivery system may further contain compound for treating a subject in need thereof. The compound may be an oligonucleotide, such as iRNA, polypeptide, such as an antibody, or a small molecule to be delivered to a subject in need thereof.

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

DNA templating is a powerful way to control dye packing—it can be used to bring molecules within 3 Å of one another. Yet, there are few studies in this field attempting to control dye packing on an even finer, e.g., sub-Å, scale. As J_{m,n} is inversely proportional to the cube of the distance between the dyes, we postulate that promoting dense dye packing will increase J_{m,n}. Dense dye packing can be achieved by promoting favorable intermolecular interactions between dyes and inhibiting such interactions between the dyes and their environment. Intermolecular interactions are embodied by dye structural properties such as hydrophobicity, electronic factors (which include dipolar and dispersion forces), and sterics.

Influence of Dye Sterics on τₚ via Φ_{F}

Excited-state (ES) quenching (i.e., small τₚ) in dye aggregates (e.g., particularly in H-aggregates) is important to overcome to effectively utilize quantum entanglement. To circumvent small τₚ, the dye structure and linkers between the dye and nucleotide will be modified to prevent H-aggregate packing, and limit dye-dye intermolecular motion (i.e., dye-dye sliding), which also facilitates ES quenching. Dye aggregates may be evaluated based on their Φ_{F}, whose value is directly proportional to τₚ; a higher Φ_{F} indicates suppressed nonradiative decay pathways and an increased τₚ. The steric hindrance between dyes in a dye aggregate will circumvent H-aggregation, limit dye-dye intermolecular motion, and enhance Φ_{F} (and τₚ). Substituting a compound, such as mesityl rings, onto the dye increases Φ_{F} due to the loss of rotational torsion caused by the compound. The compound may also lock the dyes in place. For example, a mesityl ring substituted onto a dye may hold the dye between the two methyl groups (FIG. 1A, top).

The effects of linker lengths on Φ_{F}, τₚ, and τ_{d} will also be measured as the length of the linker will enable turning of the orientation between two dyes (FIG. 1A, bottom).

### Example 2

Steric hindrance will also be measured on dye with not only a rigid linker between the dye and the nucleotide, but also two dyes bridged together using a rigid bridge. The structure of the bridge will be altered to meet the following requirements: (1) the bridge should be rigid to prevent the chromophores' conformational rotation; (2) does not form a conjugated system with the chromophores; (3) should not be too short to avoid mixing of the chromophores' ground states; and (4) should not be too long to allow the transition dipole moments to couple (for example, see FIG. 1b). The optical properties, including absorption, CD, Φ_{f}, and aggregation behavior on nucleotides of dye dimers and monomers will be characterized.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

The inventions being thus described, it will be obvious that the same may be varied in many ways. Such variations are not to be regarded as a departure from the spirit and scope of the inventions and all such modifications are intended to be included within the scope of the following claims.

### Example 3

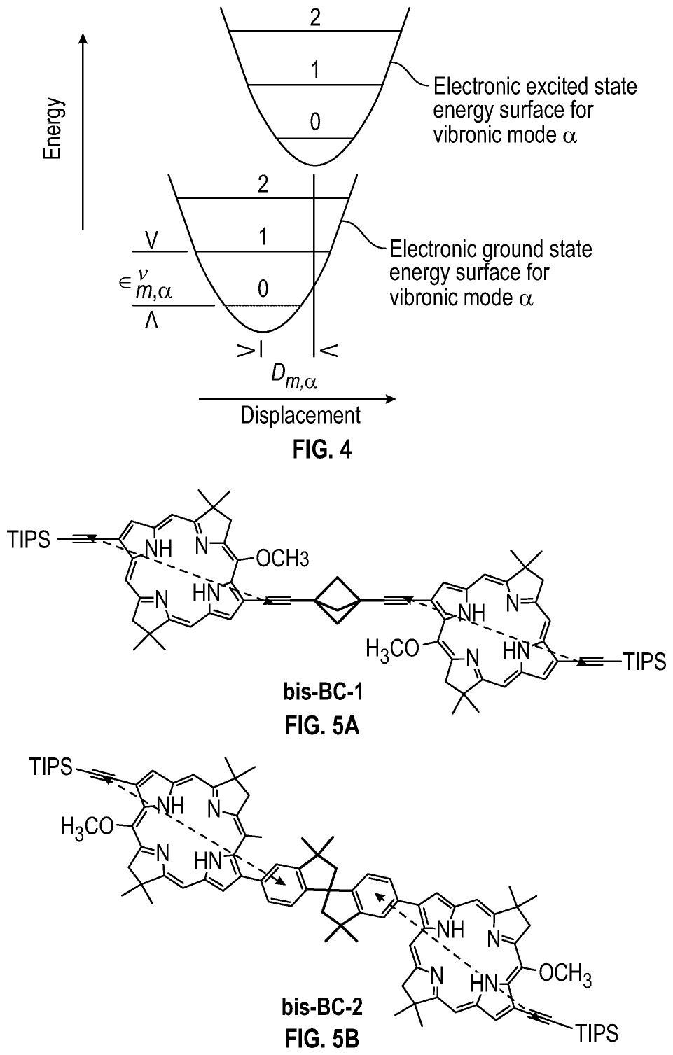

The molecular geometry of Bis-BC-1 and Bis-BC-1 was optimized using Density Functional Theory (DFT) calculations. The geometry of Bis-BC-1 afforded an end-to-end orientation of constituent long wavelength (or Q_{y}) transition dipole moments (TMD) (FIG. 4). The geometry of Bis-BC-2 afforded an end-to-end orientation of constituent transition dipole moments with some obliquity (FIG. 4). The J_{1,2} values (Table 3) were determined according to the following equations:

$${J}_{1 , 2} = {J}_{o} ( \frac{1}{\vert {r}_{1} - {r}_{2} \vert} - \frac{1}{\vert {r}_{1} - {s}_{2} \vert} - \frac{1}{\vert {s}_{1} - {r}_{2} \vert} + \frac{1}{\vert {s}_{1} - {s}_{2} \vert} ) \\ {J}_{o} = \frac{{\vert μ \vert}^{2}}{4 π {ϵ}_{0} {n}^{2} {l}_{1} {l}_{2}}$$

Where μ—the magnitude of constituent transition dipole moment calculated for the chromophores constituting dyes bis-BC-1 and bis-BC-2.

∈₀ is the permittivity of the vacuum (∈₀=8.85×10⁻¹²m⁻³kg⁻¹s⁴A²), ∈ᵣ is the relative dielectric constant of the medium, r₁ and s₁ are the location of the “+” and “−” charges on constituent chromophore 1 and similarly for constituent chromophore 2; l is the distance in meters between the two point charges on a given molecule; n is the index of refraction of the medium.

TABLE 1
Excitonic coupling strength in bis-BC dyes.

| | ¹Q_{y} TDM of constituent | |
|---|---|---|
| Dye | chromophore, debye | J_{1, 2} meV |
| Bis-BC-1 | 5.59 | 6.79 |
| Bis-BC-2 | 7.54 | 14.29 |

¹calculated in water as a solvent

### Conclusory Matters

From the foregoing, it can be seen that the present disclosure accomplishes at least all of the stated objectives.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. A rigid dye system, comprising:
a linker;
a first dye; and
a nucleotide.

2. The rigid dye system of claim 1, wherein the linker is a cyclic or acyclic, substituted or unsubstituted C2 to C30 alkyne.

3. The rigid dye system of claim 1, wherein the linker is substituted.

4. The rigid dye system of claim 3, wherein the substitution is a benzene; the dye is bonded to the benzene, and the number of benzenes ranges from about 1 to about 20.

5. The rigid dye system of claim 3, wherein the linker is acetylene.

6. The rigid dye system of claim 1, wherein the dye is substituted.

7. The rigid dye system of claim 6, wherein the substitution is an aryl.

8. The rigid dye system of claim 7, wherein the aryl is a methyl substituted benzene or a mesityl ring.

9. The rigid dye system of claim 1, wherein the first dye is a xanthene derivative, a cyanine derivative, a squaraine derivative or ring-substituted squaraine, a naphthalene derivative, a coumarin derivative, an oxadiazole derivative, an anthracene derivative, a pyrene derivative, an oxazine derivative, an acridine derivative, an arylmethine derivative, a dipyrromethane derivative, or a tetrapyrrole derivative.

10. The rigid dye system of claim 9, wherein the first dye is a tetrapyrrole derivative.

11. The rigid dye system of claim 9, wherein the first dye is a bacteriochlorin.

12. The rigid dye system of claim 1, wherein the nucleotide is RNA, DNA, LNA, PNA, BNA, or UNA.

13. The rigid dye system of claim 1, further comprising a bridge bonded to a second dye.

14. The rigid dye system of claim 1, wherein the bridge is a substituted or unsubstituted diamondoid, a bicyclic compound, an adamantane, and/or a substituted or unsubstituted cyclic or acyclic C2 to C30 alkane, alkylene, alkene, or alkyne.

15. The rigid dye system of claim 1, wherein the first dye and second dye are configured to have independent transition dipole moments.

16. A bridged dye aggregate comprising:
a first dye;
a bridge;
a bridged dye; and
a rigid linker.

17. The bridged dye aggregate of claim 16, wherein the bridge is covalently bonded between the first dye and the bridged dye.

18. The bridged dye aggregate of claim 16, wherein the bridge is a substituted or unsubstituted diamondoid, a bicyclic compound, an adamantane, or a substituted or unsubstituted cyclic or acyclic C2 to C30 alkane, alkylene, alkene, or alkyne.

19. The bridged dye aggregate of claim 16, wherein the first dye and bridge dye are configured to have independent transition dipole moments.

20. The bridged dye aggregate of claim 16, wherein the bridge dye is a population of dyes from about 1 to about 10,000 dyes.
